(19) United States
(12) Patent Application Publication
(10) Pub. No.: US 2010/0065062 A1
(43) Pub. Date: Mar. 18, 2010

(54) Temporary pharyngeal airway

(75) Inventors: Govind R.C. Rajan, Ballwin, MO (US); Marshall T. Denton, Salt Lake City, UT (US); Perry W. Croll, Salt Lake City, UT (US); Mark A. Christensen, Salt Lake City, UT (US); Timothy R. Wolfe, Salt Lake City, UT (US); J. Michael Brown, Salt Lake City, UT (US); Huy N. Tran, Riverton, UT (US)

Correspondence Address: TRASKBRITT, P.C., P.O. BOX 2550, SALT LAKE CITY, UT, 84110, US

(73) Assignee: Wolfe Tory Medical, Inc., Salt Lake City, UT (US)

(21) Appl. No.: 12/586,098

(22) Filed: Sep. 17, 2009

Related U.S. Application Data
(60) Provisional application No. 61/097,845, filed on Sep. 17, 2008.

(51) Int. Cl. A61M 16/04

(52) U.S. Cl. 128/207.14

(57) ABSTRACT

A temporary airway that can be used to facilitate an intubation procedure. Desirably, the temporary airway is removable from an installed intubation conduit without requiring disassembly of the intubation system and interrupting breathing treatment of the patient. Certain embodiments include an intubation lumen, and a treatment lumen. The intubation lumen is configured to assist in guiding an intubation conduit into operable position in a medical patient. The treatment lumen is operable to apply an anesthetic agent, and/or treatment fluids, including gas, such as Oxygen or compressed air. Certain treatment lumens are associated with a fluid dispersion nozzle, most preferably an atomizing nozzle.

### RELATED APPLICATIONS

This application claims the benefit of the filing date of U.S. Provisional Patent Application Ser. No. 61/097,845, filed Sep. 17, 2008, for “TEMPORARY AIRWAY”, the entire contents of which are hereby incorporated by this reference.

### THE FIELD OF THE INVENTION

The present invention relates to devices adapted to facilitate respiration of an awake or unconscious medical patient.

### RELATED ART

Tooling has been developed to facilitate insertion of one or more tube into a medical patient during an intubation procedure. Known tooling operable to facilitate intubation includes introducers and temporary airways. An introducer has a distally curved blade and a manipulating handle that is proximally protruding from its intersection with the blade. The entire introducer is transversely very stiff to permit its use as a path-clearing tool. A medical practitioner grasps the handle and inserts the blade into the mouth of a patient to manipulate soft tissue effective to clear a passageway to facilitate installing an intubation tube into a medical patient. The blade serves as a guide for the subsequently inserted tube. Due to its permanently affixed and proximally protruding handle, an introducer cannot be left unattended in the mouth of the patient. An inadvertent blow on the handle could cause damage to internal portions of the patient.

In contrast to an introducer, a fully installed temporary airway disposes no significant amount of substantially rigid structure protruding from the mouth of a medical patient. A temporary airway may be installed in the mouth and pharynx of an awake or unconscious medical patient. In one exemplary use, a temporary airway may be installed in a patient that has experienced mandibular, oral cavity, or pharyngeal trauma. The temporary airway functions as a breathing path by providing a conduit for air to flow past damaged or otherwise potentially blocking tissue, and may be left in place and unattended. Desirably, the temporary airway also provides guide structure to facilitate intubation of the patient.

One temporary airway known as a Williams Airway Intubator is commercially available from Anesthesia Associates, Inc. having a place of business located in San Marko, Calif. The Williams Airway Intubator carries markings indicating that it is manufactured in Canada. Such a temporary airway is structured to provide a closed-tube proximal portion, and an open-tube distal portion. The open-tube portion essentially provides a curved spatula that is shaped to approximate structure upstream of the patient's tracheal entrance. The spatula also provides a guide surface effective to orient a tube during an intubation procedure. Because the closed-tube portion is a circumferentially unbroken conduit, the temporary airway must be removed from the patient by sliding the airway proximally along any installed intubation tubing. Such a removal procedure undesirably requires disassembly of breath-supplying tubing to permit removal of the temporary airway from encircling engagement around that tubing.

Another temporary airway is known as the Berman Intubating Pharyngeal Airway. A commercially available version is packaged by Vitalsigns, Inc., which company has the web site address www.vital-signs.com. The packaging of one recently obtained embodiment carries U.S. Pat. Nos. 4,054,135; 4,067,331; 4,068,658; and 4,069,820, the disclosures of which are all hereby incorporated in their entirety by this reference. As disclosed in the aforementioned patents, the Berman Intubating Pharyngeal Airway may be structured in various ways to permit its removal from an intubated patient without requiring disassembly of the breath-supplying intubation tubing.

Temporary airways have been in use for a long period of time, as evidenced by U.S. Pat. No. 2,599,521 titled “Respiratory device”, which issued Jun. 3, 1952, to R. A. Berman. Improvements have been made on an on-going basis, as evidenced by the other previously mentioned United States patents. However, long-felt needs remain that still have not been addressed by known devices. For one example, insertion of a temporary airway into an awake patient typically requires application of an anesthetic agent to overcome the patient's automatic gag reflex. Such anesthetic application currently requires assembly of tools such as swabs, or other fluid-application devices, which complicates the procedure, and may clutter the care environment. For another example, potentially life-saving gas, such as pure Oxygen, may be administered to a patient through presently available temporary airways only subsequent to at least substantial completion of an intubation procedure. That is, application of Oxygen must wait for installation of the airway, and subsequent intubation to supply the gas. It would be an advance in the state-of-the-art to provide a temporary airway that solves one or more of such long-felt needs, or otherwise provides an improved temporary airway.

### BRIEF SUMMARY

The invention may be embodied as a temporary airway that can also be used to facilitate an intubation procedure. An exemplary temporary airway includes a body extending from a proximal end to a distal end. The body is shaped in general accordance with structure of a human mouth and pharynx, and may be formed in a plurality of sizes to accommodate small children through large adults. A wall of the body substantially encloses an intubation lumen that can be used to assist in guiding intubation tubing into an installed position in a medical patient. The body is desirably sufficiently resistant to collapse such that the patient's tongue, or other tissue, cannot obstruct the intubation lumen. Therefore, the empty intubation lumen may serve as a temporary airway conduit through which the patient may breath.

Desirably, the body's wall is configured to permit extraction of the airway from the mouth of an intubated patient without requiring disassembly of any installed intubation apparatus. One workable arrangement provides a body with a slot, or other opening arrangement, that permits passage of installed intubation tubing in a transverse direction through the wall. Sometimes, the slot may be configured to enlarge effective to permit transverse passage of intubation tubing. For example, a flexible wall may be bent out-of-the-way to permit removing a temporary airway from substantially encircling engagement around an intubation tube.

At least one treatment lumen is desirably associated with the body. A treatment lumen may be used to dispense a variety of treatment substances, nonexclusively including anesthetic agent and Oxygen. In certain cases, a pigtail, or length of tubing, may be associated with a treatment lumen or channel and arranged to protrude proximally from the temporary airway. Such a tubing desirably includes one or more connector capable of placing a treatment substance into fluid communication with the tubing. Certain connectors may place a plurality of treatment substances into fluid communication with the same length of tubing. A plurality of conduits may be associated with a body of an airway, if desired.

In some currently preferred embodiments, the body carries a treatment channel, in which one or more treatment conduit may be trapped to resist transverse displacement of the conduit(s) from within the channel. Because the body may be formed from a material that resists adherence of adhesive, axial motion of a treatment conduit relative to the body is sometimes resisted by one or more stopper. An operable stopper may be formed from an adhesive that adheres to a treatment conduit and then forms a structural interference with a portion of the treatment channel. One currently preferred embodiment of a temporary airway has a treatment channel configured to capture a pair of treatment conduits. The same principles of operation encompass one or more multi-lumen treatment conduit.

It is currently preferred to provide a temporary airway with a treatment lumen capable of dispensing anesthetic agent during installation of the airway into the mouth of a medical patient. Desirably, the treatment lumen expels anesthetic agent through a dispersion nozzle effective to spread anesthetic agent approximately evenly over an area. A currently preferred such nozzle is an atomizing nozzle. The most preferred atomizing nozzle is affixed to a treatment conduit with a coupling that forms a lap joint with both the conduit and a length along an outside diameter of the body of the atomizer.

Typically, a mouth guard is affixed at the proximal end of the body, and is configured to resist over-insertion of the body into the mouth and pharynx of the patient. Sometimes, a removable proximally-protruding handle may be provided to assist in installing a temporary airway.

### BRIEF DESCRIPTION OF THE DRAWINGS

In the drawings, which illustrate what are currently regarded as the best modes for carrying out the invention, and in which similar structures in different views are designated with like numerals:

FIG. 1 is a left side view in elevation of a temporary airway assembly that is constructed according to certain principles of the instant invention;

FIG. 2 is a front view in perspective of a temporary airway body portion of the assembly illustrated in FIG. 1;

FIG. 3 is an axially-oriented cross-section view in perspective of the body illustrated in FIG. 2;

FIG. 4 is a front view in elevation of the body illustrated in FIG. 2;

FIG. 5 is a rear view in elevation of the body assembly illustrated in FIG. 2;

FIG. 6 is a top view of the body illustrated in FIG. 2;

FIG. 7 is a bottom view of the body illustrated in FIG. 2;

FIG. 8 is a right side view in elevation of a currently preferred temporary airway assembly that is constructed according to certain principles of the instant invention;

FIG. 9 is a left side view in elevation of the body portion illustrated in FIG. 8;

FIG. 10 is a rear view in elevation of the body portion illustrated in FIG. 8;

FIG. 11 is front view in elevation of the body portion illustrated in FIG. 8;

FIG. 12 is a bottom view of the body portion illustrated in FIG. 8;

FIG. 13 is a top view of the body portion illustrated in FIG. 8;

FIG. 14 is a top side view in perspective of view of the body portion illustrated in FIG. 8;

FIG. 15 is a view in perspective, similar to the perspective in FIG. 14, of optional portions of the temporary airway that have been theoretically extracted from the assembly illustrated in FIG. 8;

FIG. 16 is a side view in perspective of an atomizer assembly that is workable in certain embodiments structured according to certain principles of the instant invention;

FIG. 17 is a cross-section side view of the atomizer illustrated in FIG. 16;

FIG. 18 is a proximal end view looking into the body of the atomizer illustrated in FIG. 16; and

FIG. 19 is a side view of an optional and removable handle that may be used with a temporary airway structured in accordance with certain principles of the instant invention.

### DETAILED DESCRIPTION OF THE ILLUSTRATED EMBODIMENTS

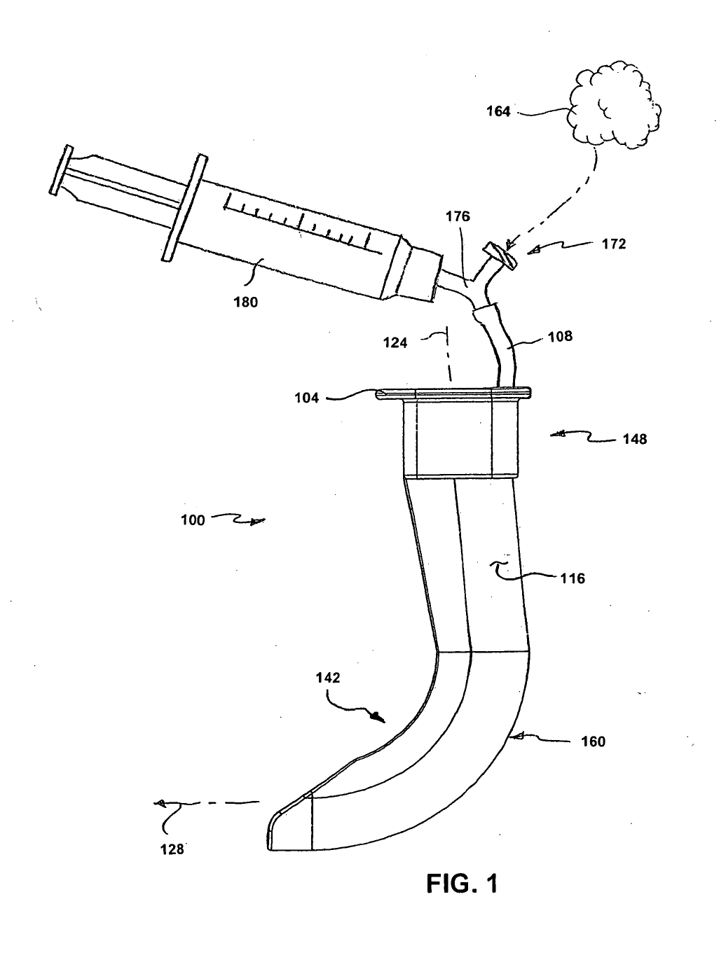

The present invention may be structured to provide a temporary airway for a medical patient. A first exemplary embodiment structured according to certain principles of the instant invention is indicated generally at 100 in FIG. 1. A temporary airway may be inserted into the oral cavity of a trauma patient in-the-field, and can be left in place and unattended for an extended period of time. Such a temporary airway 100 may sometimes directly establish a breathing path and can facilitate subsequent intubation of the patient, if required.

For purposes of this disclosure, a temporary airway differs from an introducer. In contrast to an introducer, an installed temporary airway that is structured according to certain principles of the instant invention has a limited amount of structure protruding from a patient's mouth. Certain embodiments of a temporary airway 100 may be configured to dispose no structure protruding proximally from the mouth flange 104. Other embodiments of a temporary airway may include permanently attached structure configured to protrude proximally from the mouth flange, such as one or more length of tubing 108, which is transversely compliant, and therefore cannot be used as a lever to move the internally-disposed structure of an installed temporary airway effective to manipulate, or potentially damage, soft tissue.

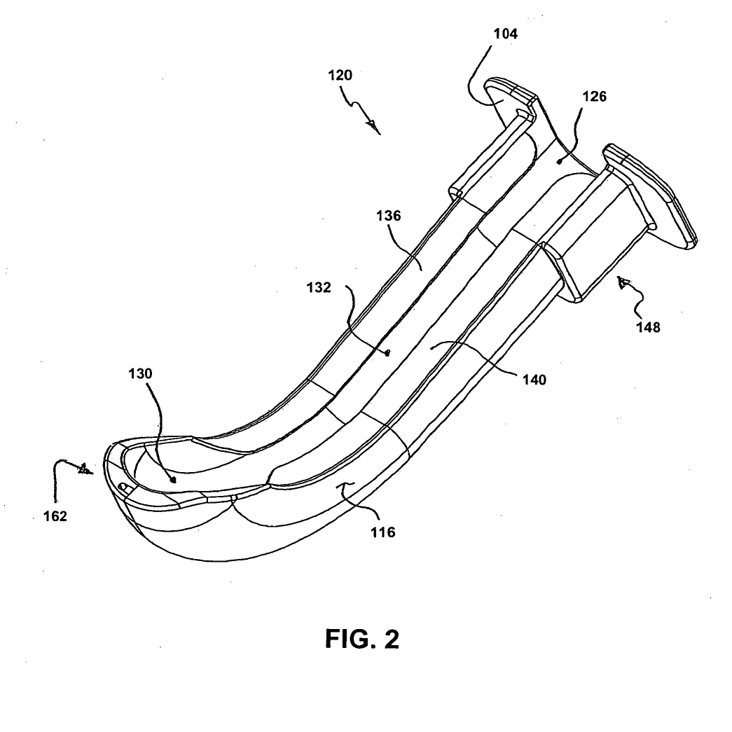

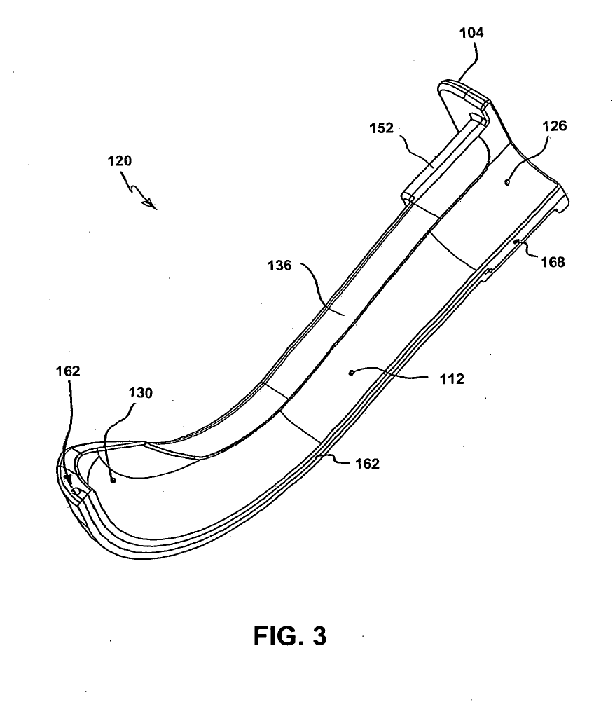

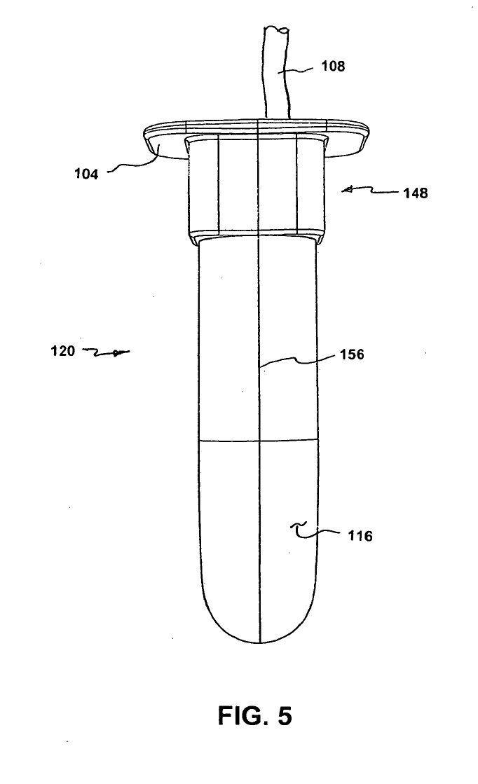

With reference to FIGS. 1-7, the illustrated temporary airway 100 provides a substantially enclosed intubation lumen 112. An intubation lumen may be defined as forming a guide path through which a tube (not illustrated), or other medical device, may be inserted into a medical patient. The substantially enclosed intubation lumen 112 provided by certain embodiments of the invention can sometimes directly establish a breathing path (an airway) through the oral cavity of certain trauma victims. An illustrated intubation lumen 112 may be regarded as being defined by, and substantially enclosed by, a wall 116 of the body portion, generally 120, of temporary airway 100.

With reference to FIG. 1, the entry axis 124 at the entrance aperture 126 for introduction of a breathing tube, or other medical instrument, is generally oriented approximately orthogonal to the discharge or aiming axis 128 disposed at the discharge aperture 130. The angle between entry axis 124 and discharge axis 128 may be characterized as the guide angle. The guide angle is typically fixed upon manufacture of the body 120, and is therefore generally not adjustable in the field. Desirably, the guide angle is effective to orient the distal end of a tube, or medical device, for its proper insertion into the desired area of a patient's internal conduit structures. The guide angle provided by temporary airway 100 is effected by a combination of factors, including without limitation: the diameter of the intubation lumen 112; the width of the open slot 132; the degree to which the right flexible wing 136 and left flexible wing 140 extend along the concave portion, generally 142, of the axially curved distal end of the body 120; and axial flexibility and cross-section size of the inserted tube or instrument.

The illustrated temporary airway 100 includes a mouth flange 104 disposed at its proximal end. The distal side of the mouth flange 104 provides a contact surface 144 that typically rests in contact with a patient's lips upon installation of the temporary airway 100. The mouth flange 104 is desirably configured and arranged to resist over-insertion of the body 120 of the airway 100 into the patient's oral cavity. Typically, the mouth flange 104 operates as a portion of a handle that a health practitioner may grip to manipulate the airway 100 during its installation into a medical patient. A finger may also be placed into entrance aperture and used as a lever during installation of an airway.

A bite guard, generally 148, is desirably disposed in distal proximity to the mouth flange 104, and is typically structured to resist imparting damage to a medical instrument (which may be installed in the intubation, or airway, lumen 112), from a patient's teeth due to an inadvertent bite-down. A wall portion 152 having sufficient thickness to resist significant deflection and/or damage from the patient's teeth may form an operable bite guard 148. As illustrated, the wall portion 152 may be reinforced, or built-up, in a local area. In other embodiments, an operable bite guard 148 may be formed from a wall of body 120 that is substantially uniform in thickness.

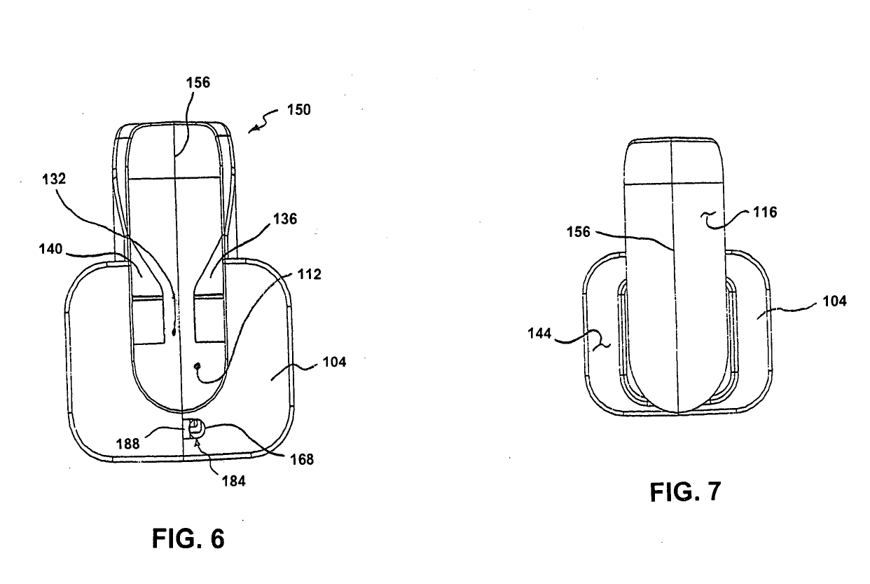

The external surface of the body 120 of a temporary airway is desirably smooth and slippery to assist in its installation and removal from the patient's oral cavity. Similarly, the guide surface internal to the airway lumen 112 is desirably smooth and slippery to assist in sliding the leading edge of a tube or instrument through that lumen. It is also desirable to provide an airway 100 with blunt corners and smooth edges, generally indicated at 150 in FIG. 6, to resist imparting trauma to the patient during insertion of the airway 100.

Body 120 may sometimes be injection molded from urethane. However, it is currently preferred to injection-mold a body from polypropylene or polyethylene, due to their ability to provide an inherently slippery surface. Operable materials of construction of the body of a temporary airway include: medical grade plastics and plastic-like materials, such as polypropylene, polycarbonate, PVC, silicone, rubber, urethane, and the like.

Illustrated body 120 of temporary airway 100 is structured as a two-part housing that is injection molded as a two-sided clamshell. Subsequent to molding, the two sides of the clamshell are joined together, forming parting line 156. A suitable connection between respective components may be formed by an adhesive, welding, or using other manufacturing techniques that are known in the art. Certain bodies 120 may alternatively include a hinged portion to permit forming the halves in the same mold, then rotating the halves approximately 90 degrees during assembly of the body.

In general, the external axially-curved convex area, generally 160, of body 120 is structured to cooperate with the shape of a patient's palette, and desirably is contiguous for at least 180 degrees around a rear circumference (that is, fully enclosed on at least the internal axially-curved concave surface), to provide a robust guide surface for an inserted tube, conduit structure, or other medical instrument, to follow. The external axially-curved concave area 142 is generally shaped in agreement with a patient's oral cavity to dispose the lumen's tube guide exit aperture 128 at the base of the patient's tongue and in an aiming orientation with respect to deeper tracheal structure.

The illustrated embodiment 100 includes an intubation lumen 112 extending through the body 120 and having an open slot 132 extending along a front wall. That is, lumen 112 includes an axially-extending front opening 132 and a substantially uninterrupted rear side. The rear side is structured to guide a conduit (e.g. intubation tubing), or medical instrument, through the oral cavity of the patient for tracheal installation of the conduit or instrument. The illustrated open slot 132 is structured to permit removal of the temporary airway 100 from the mouth of an intubated patient without requiring removal of fittings, coupling devices, or removing upstream conduit structure from fluid communication with intubation tubing prior to release of that intubation tubing from engagement within the temporary airway's lumen 112. The installed tubing may simply be passed transversely through a portion of the housing's axially extending front wall.

In one use, an intubation tube may be inserted into the proximal opening 126 of an installed temporary airway 100. The installed intubation tubing may be placed into fluid communication with a breathing apparatus to assist the patient to breath. Then, the temporary airway 100 may be removed from the patient's mouth without interrupting breathing treatment of the patient. The body 120 of the temporary airway 100, which is disposed circumferentially around the intubation tubing, may simply be stripped from engagement with the intubation tubing: either during removal of the airway 100 from seated engagement inside the patient's mouth, or subsequent to such removal.

As illustrated in FIGS. 1-7, one operable embodiment of an airway lumen 112 includes an open slot 132 with left wing 136 and right wing 140 that are each sufficiently flexible as to permit their deflection operably to widen the slot opening 132 to an extent that a conduit or instrument may be passed transversely through the opened slot 132. Such wing flexibility permits transverse extraction of an intubation tube from within the intubation lumen 112. It is within contemplation to replace the illustrated pair of wings 136, 140 with a single wing. It is further within contemplation that the wings 136, 140 may overlap one-another, to form a more fully enclosed airway lumen 112. Similarly, a single wing may be structured to overlap a portion of the body 120 to form a more fully-enclosed airway lumen 112. By “substantially enclosed” it is intended to mean structure in which a portion of the axial length of the body 120 wraps more than 180 degrees around a circumference, or other-shaped perimeter, of a portion of the length of the airway lumen 112.

Certain embodiments may optionally include one or more treatment lumen, generally 162, through which oxygen, one or more other gasses, and/or one or more treatment fluids or substances 164, may be administered. A treatment lumen 162 differs from an intubation lumen by forming a path for treatment fluids that is at least partially distinguished from the path for a breathing tube used in an intubation procedure. Sometimes, an anesthetic agent may be administered as a treatment fluid, e.g. to facilitate insertion of the temporary airway into the oral cavity of an awake patient. Also, a treatment substance 164, such as oxygen, may be dispensed during the intubation process as a bridge to prolong a time-window during which intubation may occur without causing patient mortality. It has been determined that directing a stream of Oxygen into the tracheal area of a patient may cause sufficient gas exchange in the patient's lungs as to enhance the patient's oxygen uptake without requiring the patient to inhale, or exhale.

The embodiment illustrated in FIG. 1 includes a conduit pigtail section 108 placed in fluid communication with the treatment lumen 162. As illustrated, the pigtail 108 may be disposed inside a socket 168 formed in the body 120. Such conduit section 108 may be secured in place by solvent welding, or another known manufacturing technique. It is within contemplation that a nipple may alternatively be affixed to the body 120, and the conduit installed on the nipple. It is further within contemplation that the pigtail 108 may be replaced by structure operable to form a direct connection to a device with which to dispense treatment substance into the treatment lumen, such as a portion of a luer-lock connector 172. However, it is currently preferred to include the pigtail, as the remote connection site(s), and transverse flexibility it provides is believed to be more convenient.

As illustrated, a conduit pigtail section 108 may be connected to a coupler 176 effective to place a plurality of implements, or gas/fluid sources, into fluid communication with the conduit pigtail. An operable coupler 176 includes any structure that may place one or more devices, or fluid/gas source, into fluid communication with the pigtail conduit 108. A workable coupler includes a y-connector commercially available, under part number 80386, from Qosina, having a place of business located at 150-Q Executive Drive, Edgewood, N.Y. 11717-8329, and a web site at www.qosina.com/.

The illustrated coupler 176 includes a pair of luer-lock threaded portions 172 disposed in Y-formation to receive fluid communicating devices for individual or joint fluid communication with the pigtail portion of conduit. A treatment substance 164, such as oxygen, or air, may be administered by coupling the appropriate gas source to the coupler 176. Anesthetic agent can be applied by connecting an operable supply source, such as a syringe 180, to the coupler 176.

Placing the pigtail conduit 108 (and thereby the treatment lumen 162) into fluid communication with a plurality of treatment devices permits a health practitioner to perform overlapping treatments. For example, oxygen gas may be coupled with one port of the coupler 176, and a syringe 180 loaded with anesthetic agent may be coupled with a second port of the coupler 176. In such case, a stream of Oxygen gas may be applied, and anesthetic agent may be suitably dispersed downstream from the treatment lumen discharge port under influence of the gas stream.

As perhaps best illustrated in FIG. 6, an operable treatment lumen 162 (e.g. encompassing socket 168) may be formed inside a wall of the body 120 during the manufacturing process. The illustrated treatment lumen 162 is formed, in-part, by a grove 184 disposed in one side of the body 120. Such illustrated groove 184 is sealed along the length of the body 120 upon assembly of the tongue 188, carried by the opposite body side, within the groove 184. Except for the tongue and groove structure, a first side of body 120 is structured substantially as a mirror image of its second side.

Alternative structures forming a treatment lumen 162 are within contemplation. One alternative treatment lumen 162 may be formed from a length of treatment conduit that is associated with the body 120 and configured to provide a fluid-communicating lumen extending from the proximal portion of the airway 100 and partially to, or substantially to, the distal end of the airway. Desirably, the treatment conduit would have a small diameter in comparison to the diameter of the airway or intubation lumen 112. One way to associate such a treatment conduit with an airway 100 is to simply adhere the conduit to the external surface of the body 120. A channel, or groove, may also be formed in a portion of the body, to assist in routing the treatment conduit.

In certain cases, a treatment fluid may be sufficiently dispersed simply by action of the administered gas(ses) passing across a pressure drop formed across a discharge orifice of a treatment lumen. Certain embodiments within contemplation may also include a dispersion nozzle effective to discharge fluids in a misted form from the treatment lumen's discharge orifice. It has been determined that Oxygen, air, or one or more other gasses, may be adequately administered through such an orifice effective to extend a patient's survival time prior to completion of an intubation procedure.

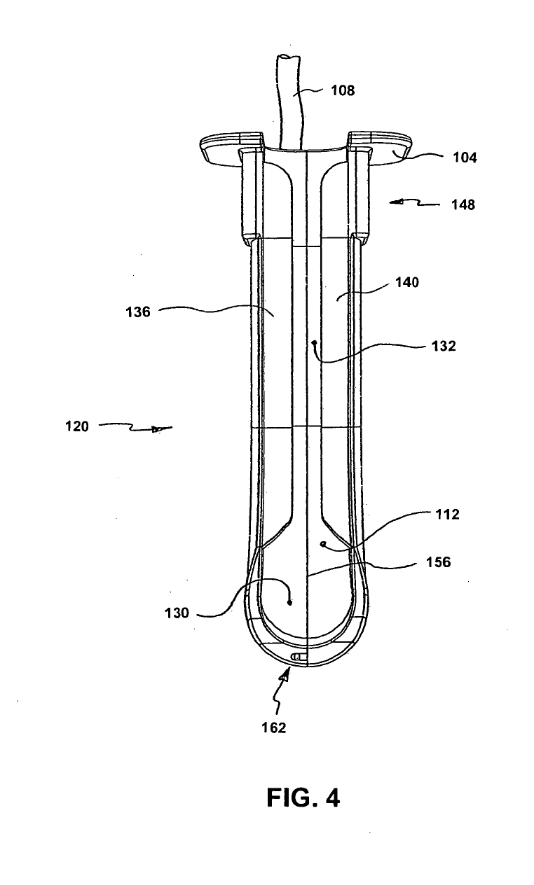

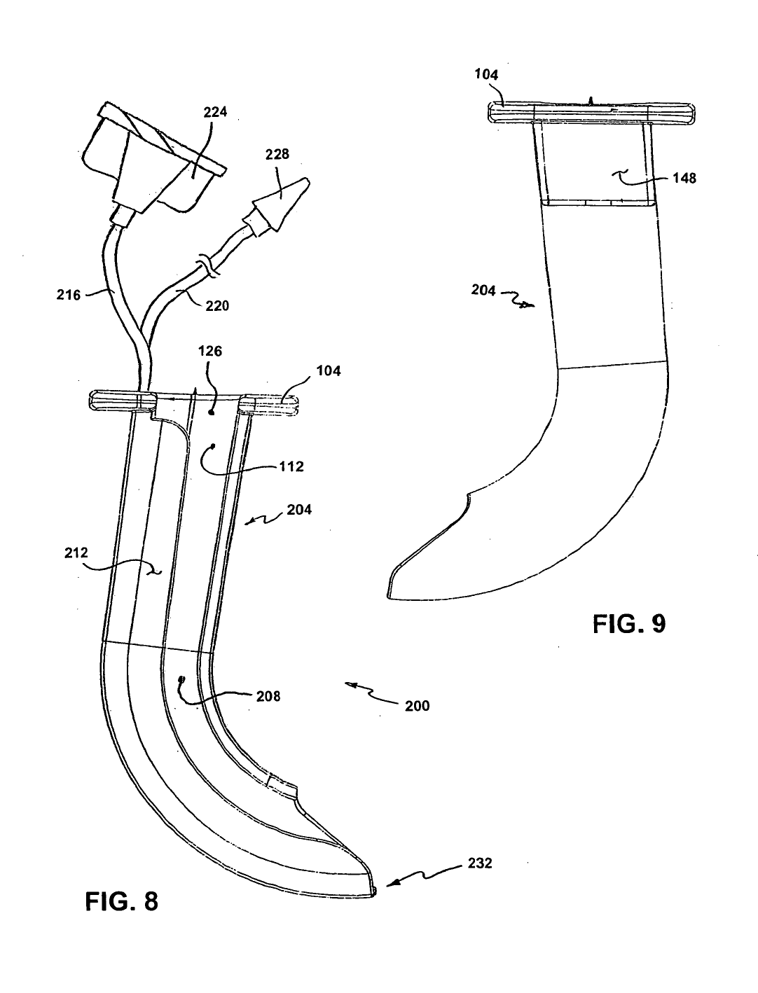

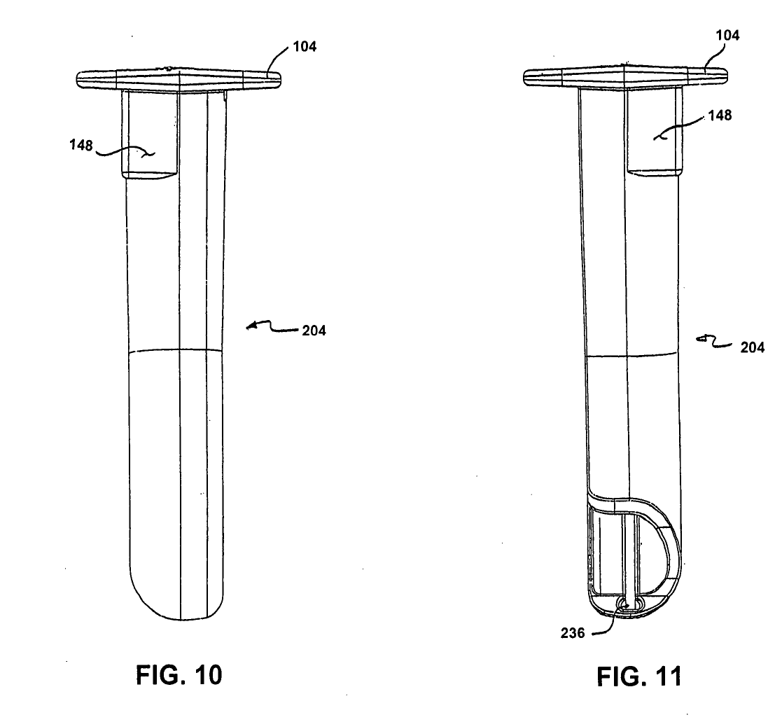

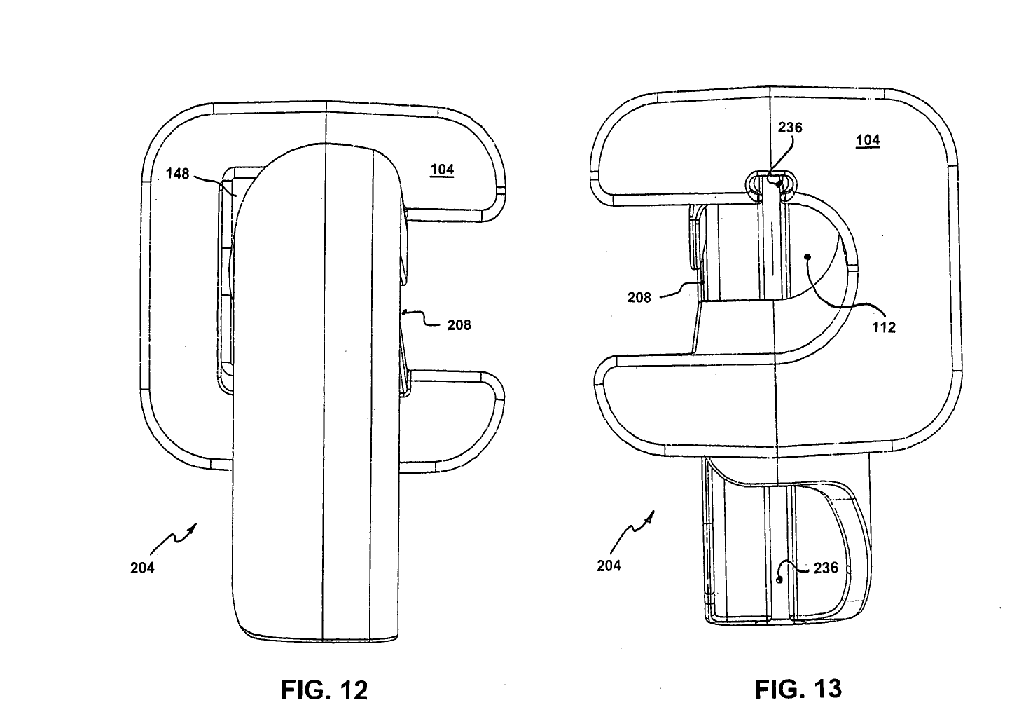

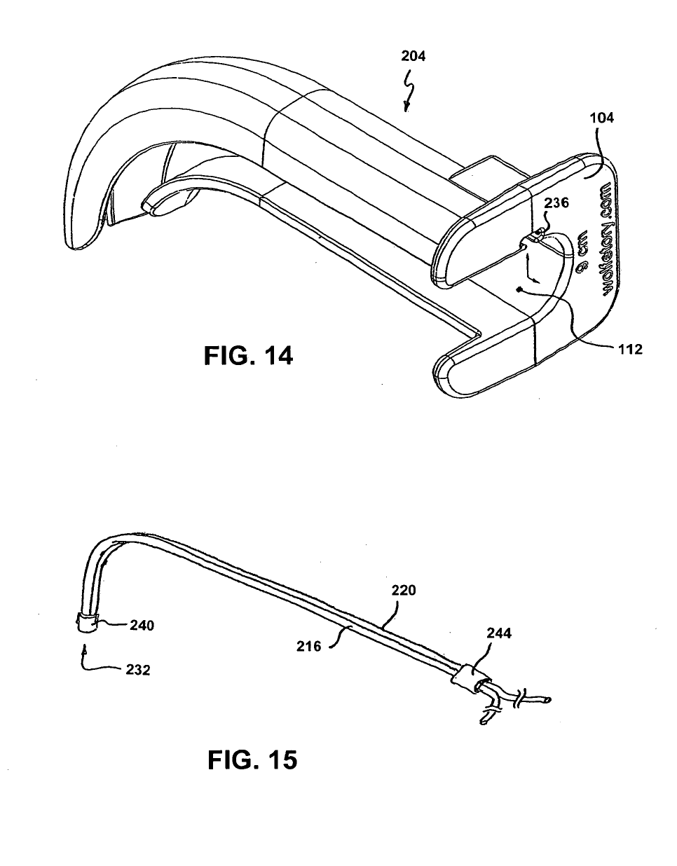

A second, and currently preferred, embodiment of a temporary airway structured according to certain principles of the instant invention is illustrated in FIG. 8, generally at 200. Temporary airway 200 includes a mouth guard 104 disposed at a proximal end of body 204. A transversely disposed side opening 208 is configured to permit removal of body 204 from substantially encircling engagement with an installed intubation tubing, similar to frontal opening 132 of body 120 (e.g. see FIG. 4). Sometimes, a wall portion 212 may be structured to permit local flexing to increase a size of opening 208 effective to facilitate extraction of an intubation tube from within lumen 112.

Still with reference to FIG. 8, a temporary airway 200 desirably includes at least one treatment lumen in addition to an intubation lumen 112. It is also within contemplation to provide a path for a fiber optic system to permit direct observation while installing a temporary airway. However, such fiber optic system is generally too expensive to include as a permanent portion of the airway.

As illustrated, temporary airway 200 includes a plurality of treatment lumens provided by treatment conduits, including conduit 216 and conduit 220. Conduit 216 is adapted to permit application of anesthetic agent during installation of the airway 200 into a medical patient. Conduit 220 is adapted to permit application of one or more treatment gas during installation of the airway 200 into a medical patient. A connector, such as luer-lock fitting 224 or hose barb 228, may be provided on a proximal end of a conduit to assist in connection to a source of fluid, such as an anesthetic agent or a gas such as Oxygen or compressed air. While it is currently preferred for the exposed axial length of a conduit to be relatively small (e.g. from less than about 1 inch to about 3 inches, or more), a conduit pigtail may have any desired length. Furthermore, any number of conduits may be provided in alternative embodiments.

Conduit 216 extends distally from a proximal end of body 204 toward a dispersion nozzle, generally 232. It is generally desirable for the dispersion nozzle 232 to be positioned at, or near, the distal end of the airway 200 to facilitate fluid dispersion. Conduit 220 extends from a proximal end of body 204 toward the distal end of body 204. Sometimes, conduit 220 may terminate at an intermediate location along a length of a body. While many ways will be apparent to one of ordinary skill-in-the-art, it is currently preferred to associate at least one treatment conduit with a body by engagement of the conduit within a channel, such as channel 236 (e.g. see FIG. 13).

It is desirable for a conduit, such conduits 216 or 220, to be formed having a relatively small outside diameter to avoid obstructing an intubation path, or requiring a large diameter in a cross-section of body 204. Illustrated conduits 216, 220 may be extruded from medical-grade plastic, and are commercially available. In a currently most preferred embodiment, each of conduits 216, 220 has an outside diameter of about 0.060 inches. It is sometimes further desirable for conduit 220 to function as an automatic regulating device to limit a flow of Oxygen to less than about 10 liters/min from a supply source at a pressure of about 50 psi. Therefore, an inside diameter of one preferred conduit 220 may be about 0.020 inches.

It is currently preferred to injection mold body 204 as a one-piece component from polyethylene or polypropylene. Such materials are capable of providing surfaces that are inherently “slippery”. However, adhesives generally do not bond well to such materials. Therefore, channel 236 is structured directly to resist removal there-from of at least one conduit in a transverse direction. In the illustrated embodiment 200, channel 236 is sized to form a slip-fit cooperating with conduits 216 and 220 to permit sliding those conduits into an installed position. Stopper structure, such as distal stopper 240 (see FIG. 15) may be affixed to a conduit to resist axial displacement of the conduit in a proximal direction with respect to the body 204. Similarly, stopper structure, such as proximal stopper 244 may be affixed to a conduit to resist axial displacement of the conduit in a distal direction with respect to the body 204.

Workable stopper structure, such as stopper 240 and stopper 244, forms a structural inference with a portion of the channel 236 to resist axial displacement of a conduit. Stopper 240 and stopper 244 can be formed by a small portion of adhesive that is applied subsequent to inserting the conduits 216 and 220 into a desired position inside channel 236. One workable adhesive is a UV-cured adhesive conventionally used in the assembly of medical products. The cured adhesive adheres well to the material forming the conduits 216 and 220. As illustrated in FIG. 15, stoppers 240 and 244 fit into oversize counter-bore sections disposed at distal and proximal ends of channel 236, respectively. It is within contemplation that a single stopper may be configured and arranged in harmony with cooperating channel structure to resist axial displacement of a conduit in both proximal and distal directions. Of course, it is to be realized that “resisting axial displacement”, and similar language, means resisting a displacement that is directed along a local length axis of at least one conduit and relative to a body.

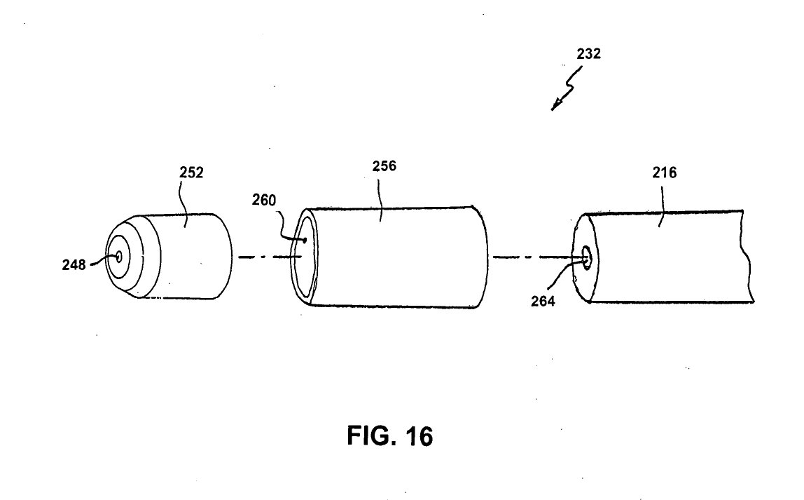

Details of an exemplary dispersion nozzle 232 are illustrated in FIGS. 16-18. It is preferred for a dispersion nozzle 232 to be operable as an atomizing nozzle, meaning that fluid dispensed through the ejection orifice 248 is dispensed substantially as a fine mist formed by very small droplets. Atomizing nozzles apply a spin to fluid prior to expelling that spinning fluid across a pressure drop formed at the ejection orifice. Higher spin rates generally correspond to a larger diameter that will be wetted by the ejected mist at a given distance from the ejection orifice.

Ejection orifice 248 is disposed at a distal end of nozzle body 252. It has been found that a workable diameter for an operable ejection orifice is about 0.008 to 0.010 inches, or so. In the illustrated embodiment 232, the diameter of an exemplary nozzle body 252 is about 0.060 inches, in harmony with the outside diameter of an exemplary and cooperating conduit 216. The exemplary nozzle body 252 may be formed by injection molding from a medical-grade plastic, such as polycarbonate, or from an alternative plastic, or plastic-like material.

Coupling 256 has a through-bore 260 with an inside diameter sized to form a slip fit with both of body 252 and conduit 216. A workable coupling 256 may be formed from relatively thin-walled tubing. One workable tubing that may be used to form a coupling 256 includes extruded polyimide tubing having a nominal outside diameter of about 0.069 inches, and a nominal inside diameter of about 0.0615 inches. A lap joint may be formed by adhesive disposed between the coupling 256 and each of nozzle body 252 and the distal end of conduit 216. Again, a workable adhesive is a UV-cured adhesive conventionally used in the assembly of medical products. Treatment fluid delivered through fluid delivery lumen 264 is therefore confined to flow distally through ejection orifice 248. Note that the body 252, conduit 216, and coupling 256 are not required to be round, although such construction is more simple.

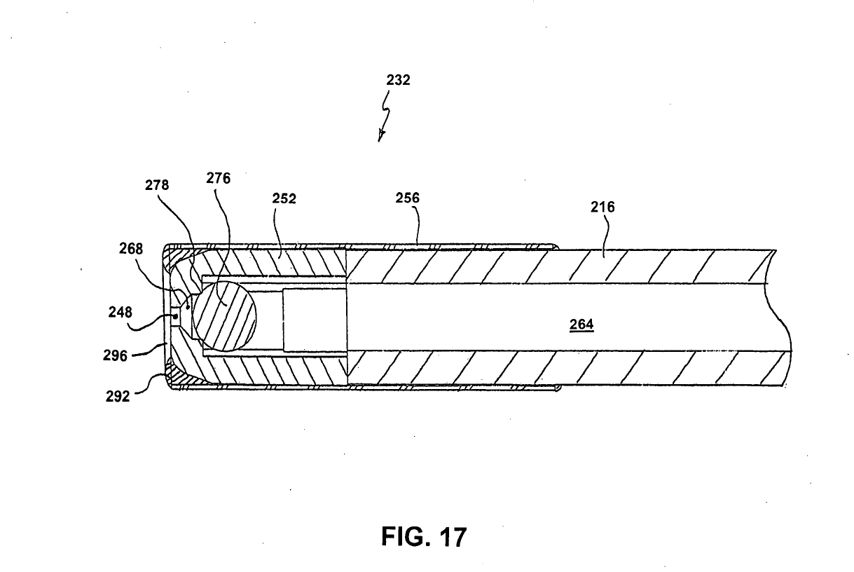

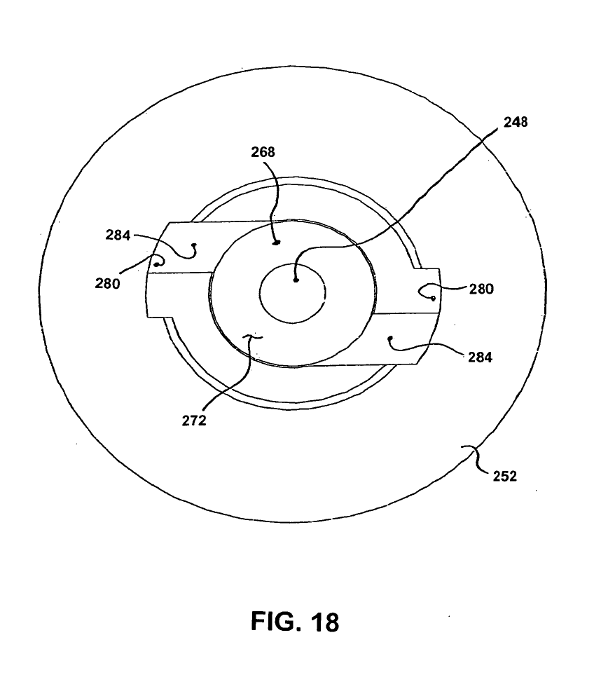

With particular reference now to FIGS. 17 and 18, a swirling chamber 268 is disposed immediately upstream from ejection orifice 248. A front wall of swirling chamber 268 includes forcing cone 272. A rear wall of swirling chamber 268 includes ball 276, although alternative components having other shapes are also workable. One exemplary workable ball 276 may be formed from stainless steel. Illustrated ball 276 has a diameter of about 1/32 inches, is configured to seat against corner 278, and may optionally be installed in a press-fit operation. Therefore, fluid delivered from lumen 264 flows past ball 276 along one or more axially-directed bypass channel 280. Fluid then enters the swirling chamber 268 through one or more turbine port 284, and develops a spin prior to being ejected through ejection orifice 248.

As illustrated in FIG. 17, it is preferred to form a wedge 292 of adhesive to act as a stopper that resists separation of the nozzle body 252 from within the coupling 256. An annular ring of adhesive 296 may be disposed to form a blunt leading edge effective to resist imparting injury to a patient during insertion of an airway.

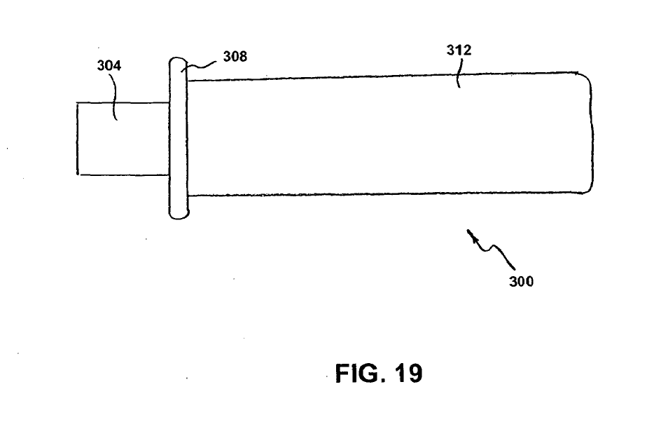

As previously mentioned, a mouth flange 104 typically operates as a portion of a handle that a health practitioner may grip to manipulate the airway 100 during its installation into a medical patient. The health practitioner may sometimes insert a gloved finger into a proximal portion of the lumen 112 to assist in manipulation of the body 120 during installation of the device into a medical patient. As one alternative, a removable handle portion, generally 300 in FIG. 19, may be provided in certain cases. Handle 300 desirably is embodied to form a lever arm that can be used to assist in installing a temporary airway, and then removed. Distal portion 304 is configured to form a slip-on plug fit inside entrance aperture 126. The cross-section of distal portion 304 is somewhat “D”-shaped, so that a torque may be applied to a temporary airway. A stop 308 may be provided to butt against the mouth guard 104 and avoid interference with conduits 216, 220, if present. Hand grip portion 312 may then be used to assist in the installation procedure.

While the invention has been described in particular with reference to certain illustrated embodiments, such is not intended to limit the scope of the invention. The present invention may be embodied in other specific forms without departing from its spirit or essential characteristics. The described embodiments are to be considered as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
a body extending from a proximal end to a distal end;
a mouth guard affixed at said proximal end and structured to resist over-insertion of said body into the oral cavity of a patient;
an intubation lumen substantially defined by a wall of said body;
said wall being structured to permit transverse passage there-through of an elongate conduit operably to permit transverse extraction of said apparatus from engagement along a length of said conduit; and
a treatment lumen associated with said body.

2. The apparatus according to claim 1, wherein:
said intubation lumen comprises an axially-extending front opening and a substantially uninterrupted rear side.

3. The apparatus according to claim 2, wherein:
said rear side is structured to guide a conduit through the oral cavity of said patient for tracheal installation of said conduit.

4. The apparatus according to claim 3, wherein:
said body comprises an axial curvature, in said rear side, configured to change an orientation of a local portion of an inserted treatment conduit effective to direct the distal end of said treatment conduit in a desired direction along a discharge axis from said body.

5. The apparatus according to claim 2, wherein:
said front opening extends the entire length of the front side of said body.

6. The apparatus according to claim 2, wherein:
said front opening is capable of enlarging to permit passage there-through of said conduit.

7. The apparatus according to claim 2, wherein:
a portion of said wall disposed adjacent to said front opening is flexible to permit enlargement of said front opening.

8. The apparatus according to claim 1, further comprising:
a pigtail conduit disposed in fluid communication with said treatment lumen.

9. The apparatus according to claim 8, further comprising:
a connector associated with said treatment lumen and structured to permit placing a dispensing device in fluid communication with said treatment conduit.

10. The apparatus according to claim 9, wherein:
said connector comprises:
a discharge port adapted for fluid communication through said pigtail to said treatment lumen; and
at least a first treatment port and a second treatment port structured for fluid communication there-through to said discharge port.

11. The apparatus according to claim 1, further comprising:
a dispersing nozzle disposed near a distal end of said treatment lumen.

12. The apparatus according to claim 11, wherein:
said dispersing nozzle comprises a fluid atomizing nozzle.

13. The apparatus according to claim 1, wherein:
said body comprises:
a first side structured to form a generally J-shaped clamshell; and
a second side structured to form a cooperating generally J-shaped clamshell; wherein:
said first side and said second side are joined together to form said rear side.

14. The apparatus according to claim 13, wherein:
said first side is structured substantially as a mirror image of said second side.

15. The apparatus according to claim 13, wherein:
said treatment lumen is defined, at least in part, by structure of a joint between said first and second sides.

16. The apparatus according to claim 15, wherein:
said joint comprises a tongue and groove arrangement.

17. The apparatus according to claim 1, wherein:
said apparatus is operable as a temporary airway.

18. The apparatus according to claim 1, wherein said treatment lumen comprises:
at least one conduit extending from a proximal end of said body toward a distal end of said body;
a channel carried by said body and structured and arranged to hold said at least one conduit effective to resist transverse separation there-between; and
first stopper structure disposed in association with a distal portion of said body, said first stopper structure being configured to resist an axial motion of said at least one conduit relative to said body.

19. The apparatus according to claim 18, further comprising:
second stopper structure disposed in association with a proximal portion of said body, said first and second stopper structure being configured to resist axial motion of said at least one conduit relative to said body.

20. The apparatus according to claim 1, wherein said treatment lumen comprises:
at least one conduit extending from a proximal end of said body toward a distal end of said body;
a channel carried by said body and structured and arranged to hold said at least one conduit effective to resist transverse separation there-between; and
stopper structure associated with said at least one conduit and configured to form a structural interference with a portion of said channel effective to resist a displacement that is directed along a local length axis of said at least one conduit and relative to said body.

21. The apparatus according to claim 1, further comprising:
a handle that may be affixed to said proximal end of said body to extend there-from in a proximal direction, said handle being configured as a lever to facilitate placing said apparatus into an installed position in a medical patient.

22. The apparatus according to claim 21, wherein:
said handle is removable.
